(19) United States
(12) Patent Application Publication
(10) Pub. No.: US 2009/0118773 A1
(43) Pub. Date: May 7, 2009

(54) MINIMAL THICKNESS BONE PLATE LOCKING MECHANISM

(76) Inventors: Anthony James, Bartlett, TN (US); Joseph M. Ferrante, Bartlett, TN (US); Paul Tornetta, Chestnut Hill, MA (US); Thomas A. Russell, Collierville, TN (US)

Correspondence Address: DIANA HOUSTON;SMITH & NEPHEW, INC., 1450 BROOKS ROAD, MEMPHIS, TN, 38116, US

(21) Appl. No.: 12/351,000

(22) Filed: Jan. 9, 2009

Related U.S. Application Data
(62) Division of application No. 11/219,850, filed on Sep. 7, 2005.
(60) Provisional application No. 60/607,630, filed on Sep. 7, 2004.

(51) Int. Cl. A61B 17/04; A61B 17/70

(52) U.S. Cl. 606/308; 606/301; 606/280; 606/286; 606/298

(57) ABSTRACT

A locking fastener for use with a bone plate. The fastener has threads on its shank to engage bone and threads on its head to engage the internal threads of the bone plate. The threads in the head may have a constant major diameter and a tapered minor diameter that creates a radial interference fit. The threads in the head may also have a variable pitch that creates an axial interference fit. The head may have a low profile to reduce soft tissue irritation.

This application claims the benefit of U.S. Provisional Application Ser. No. 60/607,630, filed Sep. 7, 2004 and titled “Minimal Thickness Bone Plate Locking Mechanism,” the entire contents of which are hereby incorporated by reference.

### FIELD OF THE INVENTION

The present invention relates generally to devices and methods for securing an orthopedic device to bone. More particularly, but not by way of limitation, the present invention relates to bone screws locked in a specific orientation within a minimal thickness bone plate. Embodiments of the present invention provide for a radial interference fit of the minor diameter of the threads on a fastener to the minor diameter of the internal threads in a bone plate. Further embodiments provide an axial interference fit through the use of a variable pitch fastener.

### BACKGROUND

Fractures are often treated with bone plates and screws which are used to secure and stabilize the fracture. Locking plates are bone plates that provide a fixed angle between the plate and a locking screw. They minimize the loosening of the screw and the plate as a result of dynamic loading or changes in the bone. Locking plates have threaded holes that engage the threads on the head of a locking screw.

Thin plates such as those used to treat peri-articular fractures present unique challenges. Peri-articular locking plates are limited in thickness by the locking mechanism. It is desirable to make peri-articular locking plates thin; however, when the plate is very thin, such as between 0.040 to 0.060 inches, typically the head of the locking screw protrudes beyond the outer surface of the plate and causes soft tissue irritation. The thin plates also reduce the locking strength of the plate because there is limited area for the typical thread configuration of the head to mate with the internal threads of the locking plate.

Accordingly, it is desirable to provide a minimal thickness bone plate locking mechanism for use with thin bone plates that allows the overall profile of the plate to remain thin and thereby reduce soft tissue irritation and yet provide for an effective fixed angle screw design. Additionally, it is desirable to have a screw that does not rely on an enlarged head to apply a generally transverse force on the outer surface of the bone plate in order to secure the screw to the plate; but rather, to have a screw that uses an interference fit within the opening of the bone plate.

### SUMMARY

Embodiments of the present invention include a fastener for use with an orthopedic device. The fastener may be, for example, a locking fastener and the orthopedic device may be, for example, a bone plate. The present invention is not limited to the thickness of the bone plate. The bone plate may be thin, especially for peri-articular applications, for example, between 0.040 to 0.060 inches, and even thinner. The bone plate, or orthopedic device generally, may be thicker, and indeed very thick, without limitation, in accordance with the present invention.

In an embodiment, the fastener is a screw. The fastener may also be a pin, peg, nail, or any other device, by any name that can generally be used to attach to an object or to connect objects. In an embodiment, the fastener has threads on its shank or shaft to engage bone and threads on its head to engage internal threads in the plate. The reference to the “head” of a fastener is intended to refer to the end, or portion of the fastener, that is closer to where force would be applied that imparts motion to the fastener. The “head” may also refer to that portion away from the portion that first enters an object. Some fasteners are commonly referred to as being “headless;” because they do not have a pronounced end portion that distinguishes the end portion from the rest of the fastener. Accordingly, the reference to a “head” of the fastener is not meant to limit the present invention in any way to a fastener with one portion that is distinguishable from the rest of the fastener.

In an embodiment, the head of a locking fastener has threads with a constant major diameter and a tapered minor diameter. The threads in the mating bone plate have a constant minor diameter. This design creates a radial interference fit between the bone plate and the expanding minor diameter of the head. The threads in the head and the plate may have multiple leads, for example, two leads, to minimize the height of the head. The head may also form part of the tapered shank. The fastener may be fixed at an angle with respect to the plate.

Another embodiment of the present invention provides for threads in the head of the fastener to have a variable pitch and the threads in the bone plate to have a constant pitch. This results in axial or in-line interference to lock the bone plate to the fastener. The locking fastener may have an interrupted thread or a continuous variable pitch thread.

An embodiment of the present invention includes a fastener for securing an orthopedic device to bone. The fastener includes a shank having a first portion and a second portion. The shank has a central longitudinal axis that passes through the first portion and the second portion. The first portion has a first end configured for contact by a driving force for moving the fastener. The second portion has a second end for engaging bone. The shank has at least one raised surface in the second portion having a crest and a distance extending radially from the central longitudinal axis to the crest. Further, the at least one raised surface in the second portion is configured to pass through an opening in the orthopedic device and to engage the bone. The first portion is configured to have an axial and/or radial interference fit within the opening in the orthopedic device.

In an embodiment, the interference fit is radial and the shank in the first and/or second portion may be tapered. In a further embodiment, there is a raised surface in the first portion and an adjacent second raised surface in the first portion wherein corresponding points on the adjacent raised surfaces in the first portion define a longitudinal distance in the first portion. There is also a raised surface in the second portion is adjacent to a second raised surface in the second portion wherein corresponding points on the adjacent raised surfaces in the second portion define a longitudinal distance in the second portion that is generally equal to the longitudinal distance in the first portion.

In another embodiment, the interference fit is axial and there is a raised surface in the first portion and an adjacent second raised surface in the first portion wherein corresponding points on the adjacent raised surfaces in the first portion define a longitudinal distance in the first portion. There is also a raised surface in the second portion that is adjacent to a second raised surface in the second portion wherein corresponding points on the adjacent raised surfaces in the second portion define a longitudinal distance in the second portion that is greater than the longitudinal distance in the first portion.

A further embodiment of the present invention is a method for fracture fixation using a locking plate, comprising the steps of reducing a fracture, placing the locking plate across the fracture and inserting the above referenced fastener through the locking plate for securing the locking plate to the bone.

### BRIEF DESCRIPTION OF THE DRAWINGS

FIG. 1 shows a cross sectional view of an embodiment of a locking fastener of the present invention.

FIG. 2a shows a cross sectional, partial view of a portion of an opening in a bone plate according to an embodiment of the present invention.

FIG. 2b shows a fastener and a plate attached to a bone.

FIG. 3 shows a cross sectional view of a further embodiment of a locking fastener of the present invention.

FIG. 4 shows a cross sectional view of a further embodiment of a locking fastener of the present invention.

FIG. 5 shows a cross sectional view of a further embodiment of a locking fastener of the present invention.

FIG. 6 shows a cross sectional, partial view of a portion of an opening in a bone plate according to an embodiment of the present invention.

FIG. 7a shows a cross sectional view of a further embodiment of a locking fastener of the present invention.

FIG. 7b shows a side view of the embodiment in FIG. 7a.

### DETAILED DESCRIPTION OF THE DRAWINGS

Embodiments of the present invention may be used to treat bone fractures, more particularly, but not by way of limitation, peri-articular fractures through the use of a thin bone plate and a minimal thickness bone plate locking mechanism. The present invention addresses the constraints that locking mechanisms place on how thin bone plates may be. Further, the invention addresses soft tissue irritation that occurs when the head of a locking fastener projects beyond the outer surface of the bone plate, particularly with respect to thin peri-articular bone plates. Still further, the invention also addresses the limitations in the locking strength that thin plates present. The locking mechanism, such as a bone screw, can be placed at a fixed angle and could be used to treat fractures such as multi-fragmentary wedge fractures or B type fractures.

Although the locking fastener of the present invention is described with reference to a bone plate used in peri-articular applications, it should be understood that the fastener may be used with any number of devices at a variety of bone sites, and may be used alone without the use of bone plates or other devices. The fasteners and orthopedic devices of the present invention may be constructed of titanium, stainless steel, or any number of a wide variety of materials possessing mechanical and biological properties suitable for attachment with bone, including absorbable material.

Reference will now be made to the figures. It should be noted that the figures are not drawn to scale. Also, a description of features that are common to multiple embodiments will not be repeated for each embodiment.

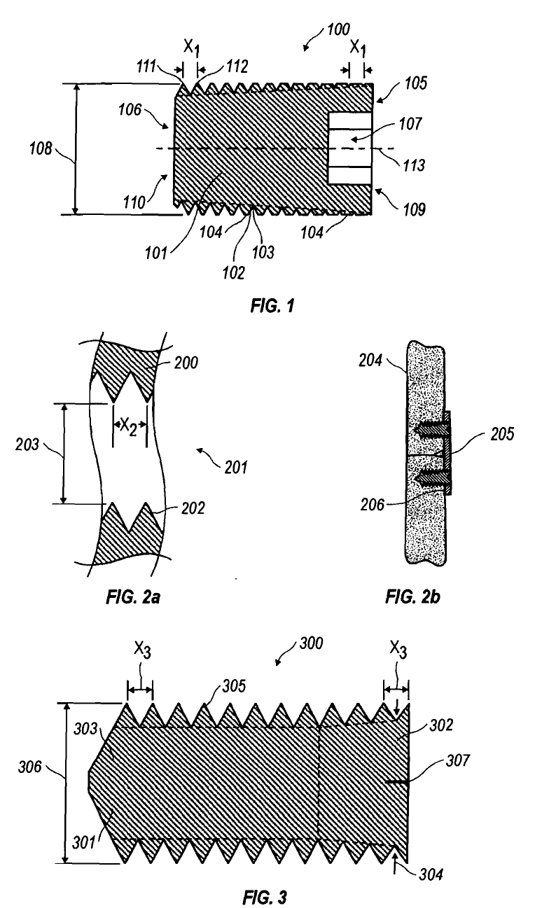

FIG. 1 shows an example of a fastener of the present invention, in this case a locking fastener 100. Locking fastener 100 has a shank or shaft 101. At least one thread 102 is arranged in a generally curved configuration, for example, a helix configuration around the shank 101. The thread 102 extends from a root 103 to a crest 104. The distance between corresponding points on adjacent thread forms is the pitch. The distance between crests 111 and 112 represents the pitch X₁. The embodiment shown in FIG. 1 has a constant pitch. The shank 101 is at least partially threaded for engaging bone and for engaging an orthopedic device, such as a bone plate. The length of the shank 101 can be selected for the particular application. The shank 101 has a first portion 105 and a second portion 106. The first portion 105 may have a first surface 109 that is configured for contact with a tool used to impart motion to the fastener 100. The first surface 109 may be configured, for example, to have a hexagonal cavity 107 that receives a correspondingly shaped tool configuration, such as a hexagonal screwdriver. It should be noted that the tool may be used to impart an axial and/or a rotational force on the fastener 100. In FIG. 1, the fastener 100 does not have a distinct transition along the shank 101 to distinguish the first portion 105 from the second portion 106. Further, in this embodiment, the first surface 109 is not raised and is at or below the outer surface of a bone plate when fully inserted, thereby reducing soft tissue irritation. The fastener 100 may be referred to as being “headless.”

The second portion 106 may have a second face 110. The second face 110 may be flat, as shown, or may have a conical shape that forms a tip. Further, the second face 110 may be shaped to have a self-tapping and/or self-drilling tip to facilitate insertion into the bone. Shank 101 can also be cannulated for receiving a guide wire. The first portion 105 has thread forms that engage an orthopedic device, such as a bone plate. The second portion has thread forms that engage bone. A thread form is any portion of the thread 102.

The largest diameter of the thread is the major diameter 108. The embodiment in FIG. 1 shows a fastener 100 wherein the largest diameter of the thread forms in the first portion 105 is generally equal to the largest diameter of the thread forms in the second portion 106. Although the largest diameter of the first portion 105 is generally equal to the largest diameter in the second portion 106, the cone-like shank 101 increases in diameter in the direction from the second portion 106 to the first portion 105. Accordingly, the smallest or minor diameter of the thread forms in the first portion 105 is larger than the minor diameter of the thread forms in the second portion 106. Because of the taper of the shank 101 and the constant major diameter 108, the distance between the crest and the root increases in the direction toward the second portion 106. This may provide greater engagement and resistance to pull out in the bone.

FIG. 2a is a partial cross sectional view of a bone plate 200 showing an opening 201 and an internal thread 202 in the opening 201. The opening 201 is oriented to allow the fastener 100 to be directed into the bone 204, as the fastener 100 passes from the outside surface 205 of the plate and then through the bone contacting surface 206 of the plate, as shown in FIG. 2b. The bone plate may have any number of openings and can have a variety of shapes, sizes, and thicknesses for use in a variety of applications. Note that the drawing is not to scale. Also, the bone plate may have smooth openings, as well as, threaded openings. The smooth openings are generally used to receive non-locking fastener and the threaded openings are generally used to receive locking fasteners. Non locking fasteners are generally used to draw the bone transversely toward the plate or to move the bone laterally through the use of compression plates.

The opening in a bone plate may be cylindrical or conical in shape. The threads in the hole may have one, two or more leads. Multiple lead threads enable multiple threads to engage while maintaining a low profile. The internal thread 202 in the opening 201 has a pitch X₂ that corresponds to the pitch X₁ of the thread 102 of the fastener 100. The internal thread 202 of the opening 201 has a minor diameter 203 that represents the smallest diameter of the thread forms of the internal thread 202. In one embodiment, the minor diameter 203 is constant in the internal thread 202. The internal thread or threads need not be formed directly on the plate, but may be formed on a separate component that lines an opening within a plate.

When fastener 100 is inserted into the opening 201 of bone plate 200 and rotates into position, the fastener 100 is able to rotate until the minor diameter 203 of the bone plate 200 interferes with the tapered shank 101 or the root 103 of the threads, thereby resulting in a radial interference fit, locking the bone fastener 100 in the bone plate 200. It can be said that the crest of the internal thread 202 contacts a root 103 of the thread 102 or contacts the tapered shank 101 of the fastener 100. It should be noted that the internal thread 202 in the bone plate and/or the opening 201 may be configured such that when the fastener 100 is inserted through the opening 201, the axis 113 along the shank 101 of the fastener 100 may be oriented in a particular direction.

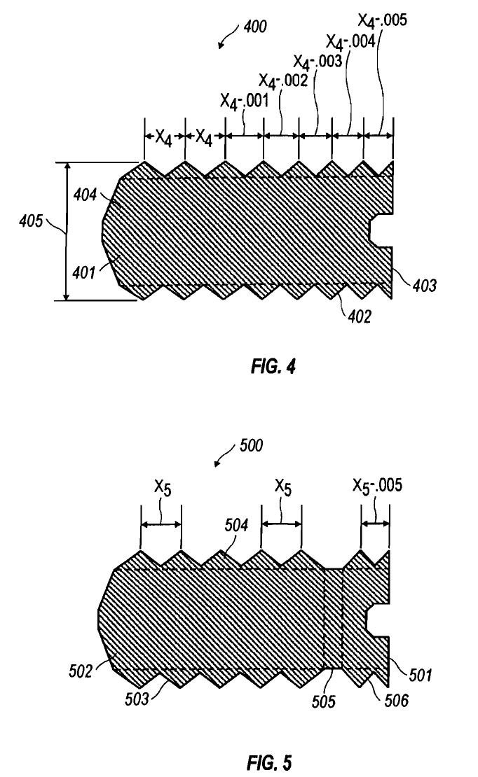

Shown in FIG. 3 is another embodiment of the present invention where the fastener 300 has a shank 301 with a first portion 302 and a second portion 303; however, in this embodiment, the diameter of the shank 301 in the second portion 303 is generally constant for most of the length of the second portion. It is in the first portion 302 that the shank 301 increases in diameter. Accordingly, the minor diameter in the thread forms in the first portion 302 is the same or larger than the minor diameter of the thread forms for most of the length in the second portion 303. The major diameter 306, of the thread forms in the first portion 302 is generally equal to the major diameter of the thread forms in the second portion 303.

With reference again to the cutaway section of the bone plate in FIG. 2a (not to scale), the fastener 300 is inserted and rotated. The fastener 300 is able to rotate until the minor diameter 203 of the bone plate 200 contacts, for example, diameter 304 of the fastener 300. Pitch X₂ of the internal thread 202 of the bone plate 200 corresponds to the pitch X₃ of the thread 305 of the fastener 300. In this embodiment, the fastener 300 is locked within the bone plate 200 at diameter 304 of the fastener 300 due to a radial interference ft.

With further reference to the bone fastener 300 in FIG. 3, other embodiments include a fastener 300 whereby the first portion 302 has multiple leads. A further embodiment includes a split collet 307 of the first portion 302 that allows for compression of the first portion 302 and forces an interference fit between the fastener 300 and plate 200. Another embodiment of the present invention includes a first portion 302 with no raised surfaces or threads on all or parts of the outer surface of the first portion 302. In an embodiment, the radial surface of the first portion 302 is smooth. An interference fit occurs because the split collet 302 allows for compression of the threadless surface of the first portion 302, causing an interference fit in the orthopedic device or plate 200. The orthopedic device may or may not have internal threads.

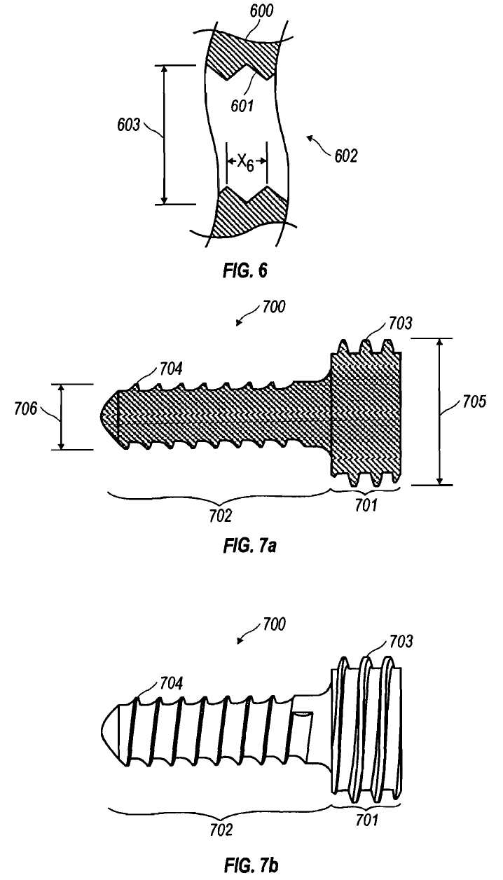

The embodiments in FIG. 4 and FIG. 5 illustrate locking through an axial interference fit. In FIG. 4, a fastener 400 has a shank 401 with a thread 402 about the shank 401. The shank 401 has a generally uniform diameter. The shank 401 has a first portion 403 and a second portion 404. The first portion 403 has thread forms of the thread 402 that engage at least one internal thread 601 of an orthopedic device, such as a bone plate 600 as depicted in FIG. 6. The shank 401 has a second portion 404 with thread forms of the thread 402 that engage bone. The major diameter 405 of the thread forms in the first portion 403 is generally the same as the major diameter of the thread forms in the second portion 404; however, the pitch of the thread 402 varies. For example, the second portion may have a pitch X₄ which is larger than, for example, pitch X₄−0.005 which is in the first portion 403. In an embodiment of the present invention, the pitch gradually decreases by 0.001 inches, for example, from X₄ to X₄−0.001, X₄−0.002, and then to X₄−0.005, as depicted in FIG. 4.

The fastener 400 is inserted into a threaded hole 602 of the bone plate 600 shown in FIG. 6. The major diameter 603 of the internal thread 601 of the bone plate corresponds to the major diameter 405 of the fastener 400. The pitch X₆ of the internal thread 601 of the bone plate 600 may correspond to a pitch X₄ of the second portion 404 of the fastener 400. As the fastener 400 rotates through the hole 602, the internal thread 601 of the bone plate 600 eventually engage and locks in place in the first portion 403 of the fastener 400. Because the pitch X₆ of the internal thread 601 corresponds to pitch X₄ of the fastener 400, as the pitch decreases on the fastener 400, an axial interference occurs to lock the fastener 400 to the bone plate 600. Other embodiments combine both axial and radial interference to achieve locking pursuant to the discussion above. For example, the fastener may have a variable minor diameter as in FIG. 1, but also have a variable pitch at the thread forms in the first portion.

FIG. 5 is a further embodiment of the present invention. The fastener 500 has a first portion 501 and a second portion 502. The first portion 501 has a thread 506 and the second portion 502 has a thread 503. The first portion 501 and the second portion 502 are separated by an area 505 on the shank 504 that does not have a thread. The pitch of the thread forms of the thread 503 in the second portion 502 may have a generally constant pitch X₅. The thread forms of the thread 506 in the first portion 501 has a pitch that is less than the pitch in the second portion 502, for example, X₅−0.005. The thread 506 in the first portion 501 is clocked to match the thread 503 in the second portion 502.

FIG. 7a is a further embodiment of the present invention. The fastener 700 has a first portion 701 and a second portion 702. The first portion 701 has a thread 703, and the second portion 702 has a thread 704. It should be noted that the first portion 701 and the second portion 702 may each have multiple threads or leads. In this embodiment, the major diameter 705 of the thread 703 in the first portion 701 is larger than major diameter 706 the thread 704 in the second portion 702. Within the first portion 701, the major diameter 705 stays constant or generally the same. Likewise, within the second portion 702, the major diameter 706 is constant or generally the same. The thread 703 in the first portion 701 is for engaging an orthopedic device, such as, a bone plate. The thread 704 in the second portion 702 is for engaging bone. As detailed above, an interference fit may be created by varying the pitch of the thread 703 in the first portion 701 and/or by varying the minor diameter of the thread 703 in the first portion 701. FIG. 7b is a side view of FIG. 7a.

It should be understood that thread pitch and the number of leads may vary in accordance with the present invention. For example, because bone plates may be very thin, one embodiment of the present invention requires a minimum of two threads on the portion of the fastener that engages the internal threads of the plate.

Additionally, the interference fit between the fastener and the plate need not be limited to only mating threads but may also encompass threads that cross and do not mate, but still provide interference and locking. Further, the interference fit may involve a smooth shank without threads.

Changes and modifications, additions and deletions may be made to the structures and methods recited above and shown in the drawings without departing from the scope or spirit of the invention and the following claims.

1-22. (canceled)

23. A fixation system for bones comprising:
a. a bone plate comprising an opening having threads, wherein the threads of the opening comprise a pitch;
b. a fastener adapted for engagement with the opening, the fastener comprising:
(i) a shaft with threads of a major diameter and configured for engaging bone; and
(ii) a head atop the shaft, the head comprising a length, a first end immediately adjacent the shaft, a second end opposite the first end, and threads having a pitch and a major diameter that is greater than the major diameter of the threads of the shaft,
wherein the pitch of the threads of the head is larger at the first end of the head than at the second end of the head and wherein the pitch of the threads along a portion of the head is less than the pitch of the threads of the opening of the bone plate such that an interference fit occurs when the head is fully inserted into the opening.

24. The system of claim 23, wherein the major diameter of the threads of the head is generally constant along the length of the head.

25. The system of claim 23, wherein the threads of the head comprise a minor diameter that is generally constant along the length of the head.

26. The system of claim 23, wherein the threads of the head comprise a minor diameter that gradually decreases along the length of the head from the second end to the first end.

27. The system of claim 23, wherein the pitch of the threads of the head gradually decreases along the length of the head from the first end to the second end.

28. The system of claim 23, wherein the pitch of the threads at the first end of the head is the same as the pitch of the threads of the opening in the bone plate.

29. The system of claim 23, wherein the portion of the head comprises the second end of the head.

30. The system of claim 23, wherein the bone plate is for a peri-articular application.

31. The system of claim 23, wherein the bone plate comprises a bone-contacting surface and an upper surface, wherein the head of the fastener does not extend beyond the upper surface when the head is fully inserted into the opening.

32. The system of claim 23, wherein the bone plate has a thickness of about 0.04 to about 0.06 inches.

33. The system of claim 23, wherein the bone plate further comprises a bone contacting surface and an outside surface opposite the bone contacting surface, and wherein the second end of the head is generally flush with the outside surface.

34. The system of claim 23, further comprising a non-locking fastener.

35. The system of claim 23, wherein the bone plate further comprises a smooth opening.

36. The system of claim 23, wherein at least two threads of the head are engaged with at least two threads of the opening.

37. The system of claim 23, wherein at least one of the bone plate and the fastener is made of an absorbable material.

38. A method for treating a fracture in a bone, the method comprising:
a. providing a bone plate;
b. placing the bone plate adjacent the bone, wherein the bone plate comprises an opening having threads comprising a pitch;
c. inserting a fastener through the opening, wherein the fastener comprises:
(i) a shaft portion for engaging bone and having threads of a major diameter; and
(ii) a head portion comprising a first end adjacent the shaft portion, a second end opposite the first end, and threads comprising a pitch and a major diameter larger than the major diameter of the shaft portion, wherein the pitch of at least some of the threads on the head portion is less than the pitch of the threads of the opening; and
d. rotating the fastener until an interference fit occurs between the head portion of the fastener and the opening of the bone plate;
wherein the interference fit comprises an axial interference fit that occurs as the at least some threads on the head portion contact the threads of the opening.

39. The method of claim 38, further comprising the step of reducing the fracture.

40. The method of claim 38, further comprising the step of placing the bone plate across the fracture.

41. The method of claim 38, further comprising the steps of providing a non-locking fastener and using the non-locking fastener to move a bone portion laterally.

42. A fixation system for bones comprising:
a. at least one bone plate comprising an opening having threads with a pitch;
b. at least one fastener adapted for engagement with the opening, wherein the fastener comprises (i) a fastener length; (ii) a plate engagement portion having first threads for engaging the opening in the plate, wherein the first threads comprise a pitch, a major diameter, and a minor diameter; and (iii) a bone engagement portion opposite the plate engagement portion and comprising second threads for engaging bone, wherein the second threads comprise a pitch, a major diameter, and a minor diameter,
wherein the pitch of at least some of the first threads is smaller than the pitch of the threads of the opening such that an interference fit occurs when the plate engagement portion is fully inserted into the opening.

43. The system of claim 42, wherein the bone plate comprises a bone contacting surface and an upper surface opposite the bone contacting surface, and wherein the plate engagement portion of the fastener does not extend above the upper surface when the plate engagement portion is fully inserted into the opening.

44. The system of claim 42, wherein the fastener is headless.

45. The system of claim 42, wherein the pitch of the first threads varies along the fastener length.

46. The system of claim 45, wherein the pitch of the first threads gradually increases along the fastener length from the plate engagement portion toward the bone engagement portion.

47. The system of claim 42, wherein the pitch of other of the first threads is substantially the same as the pitch of the threads of the opening.

48. The system of claim 42, wherein the major diameter of the first threads and the major diameter of the second threads are generally constant.

49. The system of claim 48, wherein the major diameter of the first threads and the major diameter of the second threads are approximately the same.

50. The system of claim 42, wherein the minor diameter of the first threads and the minor diameter of the second threads are generally constant.

51. The system of claim 50, wherein the minor diameter of the first threads and the minor diameter of the second threads are approximately the same.

52. The system of claim 42, wherein the minor diameter of the first threads and of the second threads gradually decreases from the plate engagement portion to the bone engagement portion.

53. The system of claim 42, wherein the fastener comprises a smooth portion between the bone engagement portion and the plate engagement portion.

54. The system of claim 53, wherein the bone engagement portion comprises a majority of the fastener length.

55. The system of claim 54, wherein the second threads comprise a generally constant pitch.

56. The system of claim 42, wherein the bone engagement portion comprises a shaft comprising the second threads and the plate engagement portion comprises a head atop the shaft, wherein the head comprises (a) a head length; (b) a first end adjacent the shaft; (c) a second end opposite the first end; and (d) the first threads, wherein the major diameter of the first threads is greater than the major diameter of the second threads.

57. The system of claim 56, wherein the pitch of the first threads is larger at the first end of the head than at the second end of the head.

58. The system of claim 57, wherein the pitch of the first threads gradually decreases along the head length from the first end of the head to the second end of the head.

59. The system of claim 56, wherein the major diameter of the first threads is generally constant along the head length.

60. The system of claim 56, wherein the minor diameter of the first threads is generally constant along the head length.

61. The system of claim 56, wherein the minor diameter of the first threads gradually decreases along the head length from the second end of the head to the first end of the head.

62. The system of claim 42 wherein the bone plate is for a peri-articular application.

63. The system of claim 42, wherein the bone plate has a thickness of about 0.04 to about 0.06 inches.

64. The system of claim 42, further comprising a non-locking fastener.

65. The system of claim 42, wherein the bone plate further comprises a smooth opening.

66. The system of claim 42 wherein at least two threads of the fastener are engaged with at least two threads of the opening.

67. The system of claim 42, wherein at least one of the bone plate and the fastener is made of an absorbable material.
